(19) United States
(12) Patent Application Publication
(10) Pub. No.: US 2004/0023977 A1
(43) Pub. Date: Feb. 5, 2004

(54) Process for making substituted thiazolyl-amino pyrimidinyl

(76) Inventors: Robert D. Larsen, Bridgewater, NJ (US); Anthony On-Ping King, Hillsborough, NJ (US)

Correspondence Address: MERCK AND CO INC, P O BOX 2000, RAHWAY, NJ, 070650907

(21) Appl. No.: 10/618,877

(22) Filed: Jul. 14, 2003

Related U.S. Application Data
(60) Provisional application No. 60/395,819, filed on Jul. 15, 2002.

(51) Int. Cl. A61K 31/506; C07D 417/14

(52) U.S. Cl. 514/252.18; 514/252.19; 544/295

(57) ABSTRACT

The present invention relates to a process for preparing substituted thiazolyl-amino pyrimidinyl piperazines, which are capable of inhibiting, modulating and/or regulating signal transduction of both receptor-type and non-receptor type tyrosine kinases.

### BACKGROUND OF THE INVENTION

The present invention relates to a process for making substituted thiazolyl-amino pyrimidinyl piperazines, which inhibit, regulate and/or modulate tyrosine kinase signal transduction, and may be used to treat tyrosine kinase-dependent diseases and conditions, such as angiogenesis, cancer, tumor growth, atherosclerosis, age related macular degeneration, diabetic retinopathy, inflammatory diseases, and the like in mammals.

Tyrosine kinases are a class of enzymes that catalyze the transfer of the terminal phosphate of adenosine triphosphate to tyrosine residues in protein substrates. Tyrosine kinases play critical roles in signal transduction for a number of cell functions via substrate phosphorylation. Though the exact mechanisms of signal transduction is still unclear, tyrosine kinases have been shown to be important contributing factors in cell proliferation, carcinogenesis and cell differentiation.

Tyrosine kinases can be categorized as receptor type or non-receptor type. Receptor type tyrosine kinases have an extracellular, a transmembrane, and an intracellular portion, while non-receptor type tyrosine kinases are wholly intracellular.

The receptor-type tyrosine kinases are comprised of a large number of transmembrane receptors with diverse biological activity. In fact, about twenty different subfamilies of receptor-type tyrosine kinases have been identified. One tyrosine kinase subfamily, designated the HER subfamily, is comprised of EGFR, HER2, HER3, and HER4. Ligands of this subfamily of receptors include epithileal growth factor, TGF-α, amphiregulin, HB-EGF, betacellulin and heregulin. Another subfamily of these receptor-type tyrosine kinases is the insulin subfamily, which includes INS-R, IGF-IR, and IR-R. The PDGF subfamily includes the PDGF-α and β receptors, CSFIR, c-kit and FLK-II. Then there is the FLK family which is comprised of the kinase insert domain receptor (KDR), fetal liver kinase-1 (FLK-1), fetal liver kinase-4 (FLK-4) and the fms-like tyrosine kinase-1 (flt-1). The PDGF and FLK families are usually considered together due to the similarities of the two groups. For a detailed discussion of the receptor-type tyrosine kinases, see Plowman et al., DN&P 7(6):334-339, 1994, which is hereby incorporated by reference.

The non-receptor type of tyrosine kinases is also comprised of numerous subfamilies, including Src, Frk, Btk, Csk, Abl, Zap70, Fes/Fps, Fak, Jak, Ack, and LIMK. Each of these subfamilies is further sub-divided into varying receptors. For example, the Src subfamily is one of the largest and includes Src, Yes, Fyn, Lyn, Lck, Blk, Hck, Fgr, and Yrk. The Src subfamily of enzymes has been linked to oncogenesis. For a more detailed discussion of the non-receptor type of tyrosine kinases, see Bolen Oncogene, 8:2025-2031 (1993), which is hereby incorporated by reference.

Both receptor-type and non-receptor type tyrosine kinases are implicated in cellular signaling pathways leading to numerous pathogenic conditions, including cancer, psoriasis and hyperimmune responses.

Several receptor-type tyrosine kinases, and the growth factors that bind thereto, have been suggested to play a role in angiogenesis, although some may promote angiogenesis indirectly (Mustonen and Alitalo, J. Cell Biol. 129:895-898, 1995). One such receptor-type tyrosine kinase is fetal liver kinase 1 or FLK-1. The human analog of FLK-1 is the kinase insert domain-containing receptor KDR, which is also known as vascular endothelial cell growth factor receptor 2 or VEGFR-2, since it binds VEGF with high affinity. Finally, the murine version of this receptor has also been called NYK (Oelrichs et al., Oncogene 8(1):11-15, 1993). VEGF and KDR are a ligand-receptor pair that play an important role in the proliferation of vascular endothelial cells, and the formation and sprouting of blood vessels, termed vasculogenesis and angiogenesis, respectively.

Angiogenesis is characterized by excessive activity of vascular endothelial growth factor (VEGF). VEGF is actually comprised of a family of ligands (Klagsburn and D'Amore, Cytokine & Growth Factor Reviews 7:259-270, 1996). VEGF binds the high affinity membrane-spanning tyrosine kinase receptor KDR and the related fms-like tyrosine kinase-1, also known as Flt-I or vascular endothelial cell growth factor receptor 1 (VEGFR-1). Cell culture and gene knockout experiments indicate that each receptor contributes to different aspects of angiogenesis. KDR mediates the mitogenic function of VEGF whereas Flt-1 appears to modulate non-mitogenic functions such as those associated with cellular adhesion. Inhibiting KDR thus modulates the level of mitogenic VEGF activity. In fact, tumor growth has been shown to be susceptible to the antiangiogenic effects of VEGF receptor antagonists. (Kim et al., Nature 362, pp. 841-844, 1993).

Solid tumors can therefore be treated by tyrosine kinase inhibitors since these tumors depend on angiogenesis for the formation of the blood vessels necessary to support their growth. These solid tumors include histiocytic lymphoma, cancers of the brain, genitourinary tract, lymphatic system, stomach, larynx and lung, including lung adenocarcinoma and small cell lung cancer. Additional examples include cancers in which overexpression or activation of Raf-activating oncogenes (e.g., K-ras, erb-B) is observed. Such cancers include pancreatic and breast carcinoma. Accordingly, inhibitors of these tyrosine kinases are useful for the prevention and treatment of proliferative diseases dependent on these enzymes.

The angiogenic activity of VEGF is not limited to tumors. VEGF accounts for most of the angiogenic activity produced in or near the retina in diabetic retinopathy. This vascular growth in the retina leads to visual degeneration culminating in blindness. Ocular VEGF mRNA and protein are elevated by conditions such as retinal vein occlusion in primates and decreased pO₂ levels in mice that lead to neovascularization. Intraocular injections of anti-VEGF monoclonal antibodies or VEGF receptor immunofusions inhibit ocular neovascularization in both primate and rodent models. Regardless of the cause of induction of VEGF in human diabetic retinopathy, inhibition of ocular VEGF is useful in treating the disease.

Expression of VEGF is also significantly increased in hypoxic regions of animal and human tumors adjacent to areas of necrosis. VEGF is also upregulated by the expression of the oncogenes ras, raf, src and mutant p53 (all of which are relevant to targeting cancer). Monoclonal anti-VEGF antibodies inhibit the growth of human tumors in nude mice. Although these same tumor cells continue to express VEGF in culture, the antibodies do not diminish their mitotic rate. Thus tumor-derived VEGF does not function as an autocrine mitogenic factor. Therefore, VEGF contributes to tumor growth in vivo by promoting angiogenesis through its paracrine vascular endothelial cell chemotactic and mitogenic activities. These monoclonal antibodies also inhibit the growth of typically less well vascularized human colon cancers in athymic mice and decrease the number of tumors arising from inoculated cells.

Viral expression of a VEGF-binding construct of Flk-1, Flt-1, the mouse KDR receptor homologue, truncated to eliminate the cytoplasmic tyrosine kinase domains but retaining a membrane anchor, virtually abolishes the growth of a transplantable glioblastoma in mice presumably by the dominant negative mechanism of heterodimer formation with membrane spanning endothelial cell VEGF receptors. Embryonic stem cells, which normally grow as solid tumors in nude mice, do not produce detectable tumors if both VEGF alleles are knocked out. Taken together, these data indicate the role of VEGF in the growth of solid tumors. Inhibition of KDR or Flt-1 is implicated in pathological angiogenesis, and these receptors are useful in the treatment of diseases in which angiogenesis is part of the overall pathology, e.g., inflammation, diabetic retinal vascularization, as well as various forms of cancer since tumor growth is known to be dependent on angiogenesis. (Weidner et al., N. Engl. J. Med., 324, pp. 1-8, 1991).

A number of compounds have been identified as inhibiting tyrosine kinase signal transduction, in particular as inhibitors of KDR. Several of these KDR inhibitors are characterized by a substituted thiazolyl-amino pyrimidinyl moiety, such as those illustrated in PCT Publication WO 02/45652 (Jun. 13, 2002).

Accordingly, a practical, efficient synthesis of substituted thiazolyl-amino pyrimidinyl piperazines is desirable and is an object of this invention.

### SUMMARY OF THE INVENTION

The present invention relates to a process for preparing substituted thiazolyl-amino pyrimidinyl piperazines, such as compounds illustrated by Formula I:

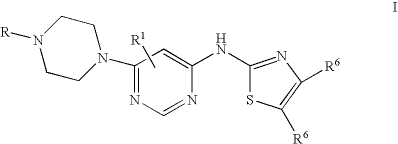

which are capable of inhibiting, modulating and/or regulating signal transduction of both receptor-type and non-receptor type tyrosine kinases.

### DETAILED DESCRIPTION OF THE INVENTION

The instant invention is related to a process for preparing substituted thiazolyl-amino pyrimidinyl piperazines, such as compounds illustrated by Formula I:

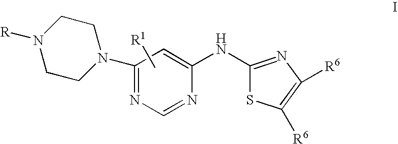

wherein

R is H, (C₁-C₆)alkyl, (C₀-C₆)alkyl-NR^{a}R^{b}, or (C₀-C₆)alkyl-C(O)N(Re)₂;

R¹ is H, or unsubstituted or substituted (C₁-C₆)alkyl;

R⁶ is independently H, phenyl, halogen, CN, or pyridyl, said phenyl and pyridyl optionally substituted with one to three substituents selcted from R⁷;

R⁷ is independently selected from:

1) Oᵣ(C═O)SNR^{a}R^{b},

2) (C═O)ᵣOₛaryl,

3) (C═O)ᵣOs-heterocyclyl,

4) halogen,

5) OH,

6) O(C₁-C₃)perfluoroalkyl,

7) (C₁-C₃)perfluoroalkyl,

8) (C═O)ᵣOₛ(C₁-C₆)alkyl,

9) CO₂H,

10) CN,

11) (C₁-C₆)alkyl-NR^{a}R^{b}, and

12) (C₁-C₆)alkyl-heterocyclyl,

wherein r and s are independently 0.0 or 1, and said aryl, heterocyclyl and alkyl are optionally substituted with one to three substituents selected from R^{d};

R^{a} and R^{b} are independently selected from:

1) H,

2) (C═O)ᵣ(C₁ C₁₀)alkyl,

3) S(O)₂R^{c},

4) (C═O)ᵣheterocyclyl,

5) (C═O)ᵣaryl, and

6) CO₂R^{c},

wherein r is 0 or 1 and said alkyl, heterocyclyl, and aryl optionally substituted with one or more substituents selected from Rd,

R^{c} is independently selected from (C₁-C₆)alkyl, aryl, and heterocyclyl;

R^{d} is independently selected from:

1) (C═O)ᵣOₛ(C₁-C₁₀)alkyl, wherein r and s are independently 0 or 1, optionally substituted with up to three substituents selected from OH, (C₁-C₆)alkoxy, halogen, heterocyclyl, CN, oxo, N(R^{e})₂ and S(O)₂R^{c},

2) Oᵣ(C₁-C₃)perfluoroalkyl,

3) (C₀-C₆)alkylene-S(O)ₘR^{c}, wherein m is 0, 1, or 2,

4) OH,

5) halo,

6) CN,

7) (C₀-C₆)alkylene-aryl, optionally substituted with up to three substituents selected from R^{e},

8) (C₀-C₆)alkylene-heterocyclyl, optionally substituted with up to three substituents selected from R^{e},

9) C(O)R^{c},

10) CO₂R^{c},

11) C(O)H,

12) N(R^{e})₂, and

13) CO₂H;

R^{e} is independently selected from:

1) H,

2) (C₁-C₆)alkyl, optionally substituted with one or more substituents selected from OH, heterocyclyl, (C₁-C₆)alkoxy, halogen, CN, oxo, N(R^{f})₂ and S(O)₂R^{c},

3) aryl, optionally substituted with one or more substituents selected from OH, heterocyclyl, (C₁-C₆)alkoxy, halogen, CN, N(R^{f})₂ and S(O)₂R^{c},

4) heterocyclyl, optionally substituted with one or more substituents selected from OH, heterocyclyl, (C₁-C₆)alkoxy, halogen, CN, oxo, N(R^{f})₂ and S(O)₂R^{c}, and

5) S(O)₂R^{c},

said heterocycle optionally substituted with one or more substituents selected from OH, (C₁-C₆)alkoxy, halogen, CN, oxo, N(R^{f})₂ and S(O)₂RC; and

R^{f} is independently selected from H, aryl and (C₁-C₆)alkyl;

which comprises the steps of:

a) reacting a compound of Formula II

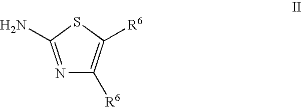

with a compound of Formula III

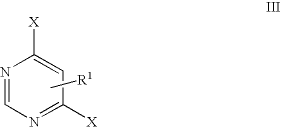

(wherein X is halo), to provide a compound of Formula IV

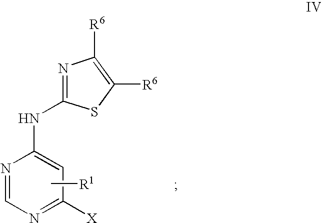

b) reacting the compound of Formula IV with a compound of Formula V

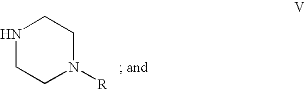

c) isolating the compound of Formula I.

A further embodiment of the above described process comprises the steps of:

a) adding the compounds of Formula II and Formula m and a phosphate to a first solvent;

b) isolating the compound of Formula IV;

c) adding the compound of Formula IV and a trialkylamine to a mixture of the compound of Formula V in a second solvent; and

d) isolating the compound of Formula I.

Another embodiment of the process for preparing a compound of Formula I comprises the steps of:

a) adding the compounds of Formula II and Formula III and a carbonate to a first solvent;

b) isolating the compound of Formula IV;

c) adding the compound of Formula IV and a trialkylamine to a mixture of the compound of Formula V in a second solvent; and

d) isolating the compound of Formula I.

A second embodiment of the instant invention is a process for preparing 2-(4-{6-[(5-cyano-1,3-thiazol-2-yl)amino]pyrimidin-4-yl}piperazin-1-yl)-N-isopropylacetamide

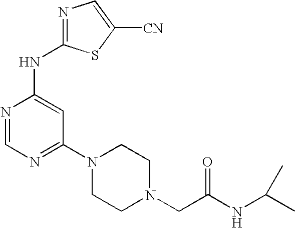

which comprises the steps of:

a) adding 2-amino-5-cyanothiazole, dichloropyrmidine, and K₃PO₄ to DMF to provide 2-[(6-chloropyrimidin-4-yl)amino]-1,3-thiazole-5-carbonitrile;

b) adding 2-[(6-chloropyrimidin-4-yl)amino]-1,3-thiazole-5-carbonitrile and triethylamine to N-Isopropyl-2-piperazin-1-ylacetamide in n-butanol; and

c) isolating 2-(4-{6-[(5-cyano-1,3-thiazol-2-yl)amino]pyrimidin-4-yl}piperazin-1-yl)-N-isopropylacetamide.

“Tyrosine kinase-dependent diseases or conditions” refers to pathologic conditions that depend on the activity of one or more tyrosine kinases. Tyrosine kinases either directly or indirectly participate in the signal transduction pathways of a variety of cellular activities including proliferation, adhesion and migration, and differentiation. Diseases associated with tyrosine kinase activities include the proliferation of tumor cells, the pathologic neovascularization that supports solid tumor growth, ocular neovascularization (diabetic retinopathy, age-related macular degeneration, and the like) and inflammation (psoriasis, rheumatoid arthritis, and the like).

The compounds of the present invention may have asymmetric centers, chiral axes, and chiral planes (as described in: E. L. Eliel and S. H. Wilen, Stereo-chemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted.

When any variable (e.g. R⁶, Ra, etc.) occurs more than one time in any constituent, its definition on each occurrence is independent at every other occurrence. Also, combinations of substituents and variables are permissible only if such combinations result in stable compounds. Lines drawn into the ring systems from substituents indicate that the indicated bond may be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched and unbranched, cyclic and acyclic saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement and may be cyclic or acyclic. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, cyclopropyl, methyl-cyclopropyl, 2,2-dimethyl-cyclobutyl, 2-ethyl-cyclopentyl, cyclohexyl, and so on. In some instances, definitions may appear for the same variable reciting both alkyl-and cycloalkyl-when a different number of carbons is intended for the respective substituents. The use of both terms in one definition should not be interpreted to mean in another definition that “alkyl” does not encompass “cycloalkyl” when only “alkyl” is used. “Alkoxy” represents an alkyl group of indicated number of carbon atoms as defined above attached through an oxygen bridge.

If no number of carbon atoms is specified, the term “alkenyl” refers to a non-aromatic hydrocarbon radical, which may be branched or unbranched and cyclic or acyclic, containing from 2 to 10 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl, cyclohexenyl, methylenylcyclohexenyl, and so on.

The term “alkynyl” refers to a hydrocarbon radical, which may be branched or unbranched and cyclic or acyclic, containing from 2 to 10 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃) CH₂CH(CH₃)Ph, and so on.

As used herein, “aryl” is intended to mean phenyl and substituted phenyl, including moieties with a fused benzo[group. Examples of such aryl elements include phenyl, naphthyl, tetrahydronaphthyl, indanyl, biphenyl, phenanthryl, anthryl or acenaphthyl. In cases where the aryl substituent is bicyclic, it is understood that attachment is via the phenyl ring. Unless otherwise indicated, “aryl” includes phenyls substituted with one or more substituents.

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl,-pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

As appreciated by those of skill in the art, “halo” or “halogen” as used herein is intended to include chloro, fluoro, bromo and iodo.

The term “heterocycle” or “heterocyclyl” as used herein is intended to mean a 5- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrathydro analogs thereof. Further examples of “heterocyclyl” include, but are not limited to the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, aziridinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

The alkyl, alkenyl, alkynyl, cycloalkyl, aryl, heteroaryl and heterocyclyl substituents may be unsubstituted or unsubstituted, unless. specifically defined otherwise. For example, a (C₁-C₆)alkyl may be substituted with one, two or three substituents selected from F, Cl, Br, CF₃, N₃, NO₂, NH₂, oxo, —OH, —O(C₁-C₆ alkyl), S(O)0-2, (C₁-C₆ alkyl) S(O)0-2-, (C₁-C₆ alkyl)S(O)0-2(C₁-C₆ alkyl)-, C₃-C₁₀ cycloalkyl, C₂-C₆ alkenyl, C₂-C₆ alkynyl, —C(O)NH, (C₁-C₆ alkyl) C(O)NH—, H₂NC(NH)—, (C₁-C₆ alkyl)C(O)—, —O(C₁-C₆ alkyl)CF₃, (C₁-C₆ alkyl)OC(O)—, (C₁-C₆ alkyl)O(C₁-C₆alkyl)-, (C₁-C₆ alkyl)C(O)₂(C₁-C₆ alkyl)-, (C₁-C₆ alkyl)OC(O)NH—, aryl, benzyl, heterocycle, aralkyl, heterocyclylalkyl, halo-aryl, halo-benzyl, halo-heterocycle, cyano-aryl, cyano-benzyl and cyano-heterocycle. In this case, if one substituent is oxo and the other is OH, the following are included in the definition: —(C═O)CH₂CH(OH)CH₃, —(C═O)OH, —CH₂(OH)CH₂CH(O), and so on.

In a preferred embodiment of the instant process for preparing a compound of Formula I, a compound of Formula II, a compound of Formula III and a phosphate or a carbonate are added to a first solvent. A compound of Formula IV and an unsubstituted or substituted amine is added to mixture of a compound of Formula V in a second solvent. Then, the compound of Formula I is isolated.

Types of phosphates that may be used in the instant process may include, but are not limited to, cesium phosphate, lithium phosphate, potassium phosphate, sodium phosphate, and the like. Types of carbonates that be may utilized may include, but are not limited to, cesium carbonate, lithium carbonate, potassium carbonate, sodium carbonate, and the like As used herein, a “first solvent” may include, but is not limited to, unchlorinated or chlorinated hydrocarbons, nitriles, ethers, polar aprotic solvents or mixtures thereof. Types of unchlorinated hydrocarbons include, but are not limited to, toluene or xylene. Types of chlorinated hydrocarbons include, but are not limited to, dichloromethane, chloroform, chlorobenzene or ODCB. Types of nitriles include, but are not limited to, acetonitrile, propionitrile, benzonitrile or tolunitrile. Types of ethers include, but are not limited to, diethyl ether, MTBE, THF, DME and DEM. Types of polar aprotic solvents include, but are not limited to, formamide, DMF, DMAC, NMP, DMPU, DMSO, and sulfolane. Preferably, the first solvent is DMF, DMAC, Toluene, Acetonitirile, or an ether. Most preferably, the first solvent is DMF or DMAC.

As used herein, the alkyl in the term “trialkylamine” is intended to include both branched and unbranched, cyclic and acyclic saturated aliphatic hydrocarbon groups having from 1 to 8 carbon atoms. Types of trialkylamines that may be used include, but are not limited to triethylamine, diisopropylethylamine, trioctylamine, dipropylethylamine, tributylamine and the like. Preferably, triethylamine is used.

As used herein, a “second solvent” may include, but is not limited to, water, alcohols, unchlorinated or chlorinated hydrocarbons, nitriles, ketones, ethers, polar aprotic solvents or mixtures thereof. Types of alcohols that can be used include, but are not limited to, methanol, ethanol, n-propanol, i-propanol, butanol or an alkoxyethanol. Types of unchlorinated hydrocarbons include, but are not limited to, toluene or xylene. Types of chlorinated hydrocarbons include, but are not limited to, dichloromethane, chloroform, chlorobenzene or ODCB. Types of nitriles include, but are not limited to, acetonitrile, propionitrile, benzonitrile or tolunitrile. Types of ketones include, but are not limited to, acetone, MEK, MIBK and cyclohexanone. Types of ethers include, but are not limited to, diethyl ether, MTBE, THF, DME and DEM. Types of polar aprotic solvents include, but are not limited to, formamide, DMF, DMAC, NMP, DMPU, DMSO, and sulfolane. Preferably, the second solvent is an alcohol, a polar aprotic solvent or an ether. More preferably, the second solvent is an alcohol.

The salts of compounds utilized in the instant processes include the conventional salts of the compounds, e.g., inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, trifluoroacetic and the like.

With respect to compounds which contain an acid moiety, a salt may take the form, for example, —COOM, where M is a negative charge, which is balanced by a counterion, e.g., an alkali metal cation such as sodium or potassium. Other pharmaceutically acceptable counterions may be calcium, magnesium, zinc, ammonium, or alkylammonium cations such as tetramethylammonium, tetrabutylammonium, choline, triethylhydroammonium, meglumine, triethanolhydroammonium and the like.

Some of the abbreviations that may be used in the description of the chemistry and in the Examples include:

| | |
|---|---|
| ACN | Acetonitrile; |
| Ac₂O | Acetic anhydride; |
| AcOH | Acetic acid; |
| AIBN | 2,2′-Azobisisobutyronitrile; |
| BINAP | 2,2′-Bis(diphenylphosphino)-1,1′ binaphthyl; |
| Bn | Benzyl; |
| BOC/Boc | tert-Butoxycarbonyl; |
| BSA | Bovine Serum Albumin; |
| CAN | Ceric Ammonia Nitrate; |
| CBz | Carbobenzyloxy; |
| CI | Chemical Ionization; |
| DBA | dibenzanthracene; |
| DBAD | Di-tert-butyl azodicarboxylate; |
| DBU | 1,8-Diazabicyclo[5.4.0]undec-7-ene; |
| DCE | 1,2-Dichloroethane; |
| DEAD | diethylazodicarboxylate; |
| DEM | diethoxymethane; |
| DIAD | diisopropylazodicarboxylate; |
| DIEA | N,N-Diisopropylethylamine; |
| DMAC | N,N-dimethylacetamide; |
| DMAP | 4-Dimethylaminopyridine; |
| DME | 1,2-Dimethoxyethane; |
| DMF | N,N-Dimethylformamide; |
| DMPU | 1,3-Dimethyl-3,4,5,6-tetrahydro-2-(1H)-pyrimidinone; |
| DMSO | Methyl sulfoxide; |
| DPAD | dipiperidineazodicarbonyl; |
| DPPA | Diphenylphosphoryl azide; |
| DTT | Dithiothreitol; |
| EDC | 1-(3-Dimethylaminopropyl)-3-ethyl-carbodiimide- |
| | hydrochloride; |
| EDTA | Ethylenediaminetetraacetic acid; |
| ES | Electrospray; |
| ESI | Electrospray ionization; |
| Et₂O | Diethyl ether; |
| Et₃N | Triethylamine; |
| EtOAc | Ethyl acetate; |
| EtOH | Ethanol; |
| FAB | Fast atom bombardment; |
| HEPES | 4-(2-Hydroxyethyl)-1-piperazineethanesulfonic acid; |
| HOAc | Acetic acid; |
| HMTA | Hexamethylenetetramine; |
| HOBT | 1-Hydroxybenzotriazole hydrate; |
| HOOBT | 3-Hydroxy-1,2,2-benzotriazin-4(3H)-one; |
| HPLC | High-performance liquid chromatography; |
| HRMS | High Resolution Mass Spectroscopy; |
| KOtBu | Potassium tert-butoxide; |
| LAH | Lithium aluminum hydride; |
| LCMS | Liquid Chromatography Mass Spectroscopy; |
| MCPBA | m-Chloroperoxybenzoic acid; |
| Me | Methyl; |
| MEK | Methyl ethyl ketone; |
| MeOH | Methanol; |
| MIBK | Methyl isobutyl ketone; |
| Ms | Methanesulfonyl; |
| MS | Mass Spectroscopy; |
| MsCl | Methanesulfonyl chloride; |
| MsOH | methanesulfonic acid; |
| MTBE | tert-butyl methyl ether; |
| n-Bu | n-butyl; |
| n-Bu₃P | Tri-n-butylphosphine; |
| NaHMDS | Sodium bis(trimethylsilyl)amide; |
| NBS | N-Bromosuccinimide; |
| NMP | N-Methyl pyrrolidinone; |
| ODCB | Ortho Dichlorobenzene, or 1,2-dichlorobenzene; |
| Pd(PPh₃)₄ | Palladium tetrakis(triphenylphosphine); |
| Pd₂(dba)₂ | Tris(dibenzylideneacetone)dipalladium (0) |
| Ph | phenyl; |
| PMSF | α-Toluenesulfonyl fluoride; |
| Py or pyr | Pyridine; |
| PYBOP | Benzotriazol-1-yloxytripyrrolidinophosphonium |
| (or PyBOP) | hexafluorophosphate; |
| RPLC | Reverse Phase Liquid Chromatography; |
| rt (or RT) | Room Temperature; |
| t-Bu | tert-Butyl; |
| TBAF | Tetrabutylammonium fluoride; |
| TBSCI | tert-Butyldimethylsilyl chloride; |
| TFA | Trifluoroacetic acid; |
| THF | Tetrahydrofuran; |
| TIPS | Triisopropylsilyl; |
| TMEDA | N,N,N′,N′-Tetramethylethylenediamine; |
| TMS | Tetramethylsilane; |
| Tr | Trityl; and |
| TsOH | P-Toluenesulfonic acid. |

The use of the process of the instant invention to prepare KDR inhibitors (such as those described in PCT Publ. WO 02/45652 (Jun. 13, 2002) is illustrated in the following schemes, in addition to other standard manipulations that are known in the literature or exemplified in the experimental procedures. These schemes, therefore, are not limited by the compounds listed or by any particular substituents employed for illustrative purposes. Substituent numbering as shown in the schemes does not necessarily correlate to that used in the claims. For example, the substitutent

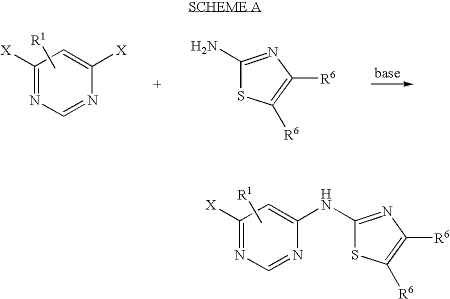

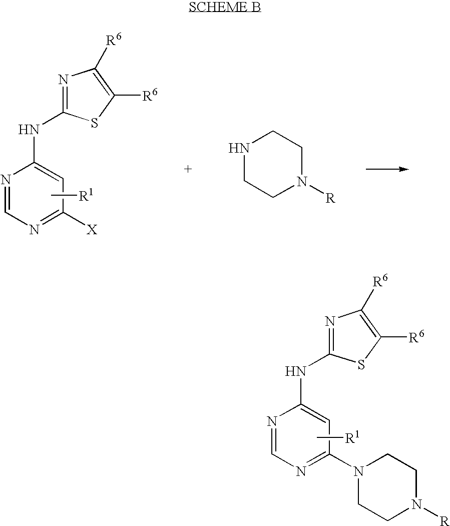

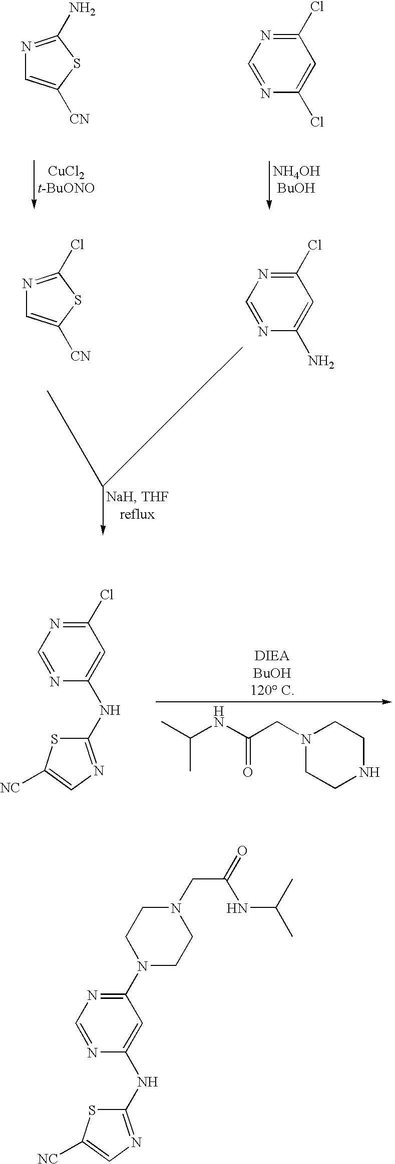

### EXAMPLES

Examples provided are intended to assist in a further understanding of the invention. Particular materials employed, species and conditions are intended to be further illustrative of the invention and not limiting of the reasonable scope thereof.

### Example 1

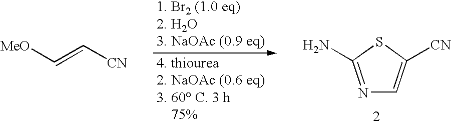

Bromine (2.88 kg, 18.0 mol) is added to a solution of 3-methoxyacrylonitrile (1.50 kg, 18.0 mole, mixture of cis-/trans-isomers) in acetonitrile (3.00 L) at 5-10° C. The mixture is aged for 20 min, then pre-cooled water (˜5° C., 12.0 L) is added and the mixture is stirred vigorously for 1 hour.

NaOAc.3H₂O, (2.21 kg, 16.2 mol, 0.90 equiv.) is added and the mixture is stirred for 15 min. Thiourea (1.51 kg, 19.80 mol, 1.10 equiv.) is added (endothermic dissolution followed by ˜10-15° C. exotherm in ˜0.5 h). The mixture is aged at 15° C. for 1.5 h, then more NaOAc.3H₂O (1.47 Kg, 0.60 equiv.) is added.

The reaction mixture is slowly heated to 60° C. over 1 h, aged for 3 h at 60° C., and cooled to 10° C.

Aqueous NaOH (10 N, 1.13 L, 0.625 equiv.) is added to adjust the pH to 3.8-4.0. After the mixture is aged for 1 h, the product is filtered, washed with water (11.5 L), and dried, giving 1.86 kg of the crude aminothiazole as-a brown solid (97A %) in 80.7% yield, corrected for 97.6 w % purity.

The crude product is dissolved in acetone (35 L) at 50° C. and treated with Darco KB-B (380 g) for 2 h. The mixture is filtered through a Solka-Floc pad and then rinsed with acetone (5 L). The filtrate is concentrated in vacuo to ˜7 L (˜5 L residual acetone).

Heptane (10 L) is added over 0.5 h and the slurry is aged for 1 h. The product is filtered and the filter cake is washed with 2:1 heptane/acetone (6 L). Drying at room temperature affords 1.72 kg of 2-amino-5-cyanothiazole (2) as a pinkish solid in 75% yield.

HPLC conditions:

| Column: | Ace-C8 4.6 × 250 mm | | |
|---|---|---|---|
| Gradient: | Time | 0.1% H₃PO₄ | Acetonitrile |
| 0 min | 95% | 5% | |
| 12 min | 80% | 20% | |

Flow rate: 1.50 mL/min
UV detection: 220 nm.

Retention times:

| | | | |
|---|---|---|---|
| Compounds | MeO—CH═CHCN | thiourea | aminothiazole |
| RT (minutes) | 5.52 (trans-) | 2.20 | 5.21 |
| | 5.78 (cis-) | | |

### Example 2

Preparation of 2-[(6-chloropyrimidin-4-yl)amino]-1,3-thiazole-5-carbonitrile (7)

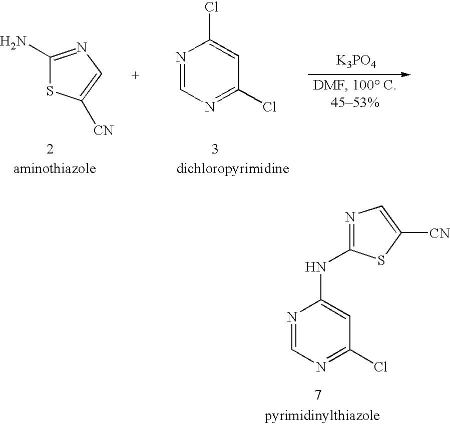

Aminothiazole 2 (1.25 g, 10 mmol), dichloropyrimidine 3 (1.49 g, 10 mmol) and K₃PO₄ (4.24 g, 20 mmol) are added to DMF (30 mL). The mixture is heated to 80° C. and aged overnight (16 h) at which time the reaction is complete. The assay yield is 45%. Water (60 mL) is added and a solution of aqueous H₃PO₄ (3 mL of 85% H₃PO₄ in 7 mL of water) is added dropwise until the pH is adjusted to ˜4. After the mixture is aged for 1 h, the precipitated black solid is filtered, washed with water and dried on the filter funnel. A 53% yield of the crude product is obtained.

### Example 3

Preparation of 2-(4-{6-[(5-cyano-1,3-thiazol-2-yl)amino]pyrimidin-4-yl}piperazin-1-yl)-N-isopropylacetamide (1)

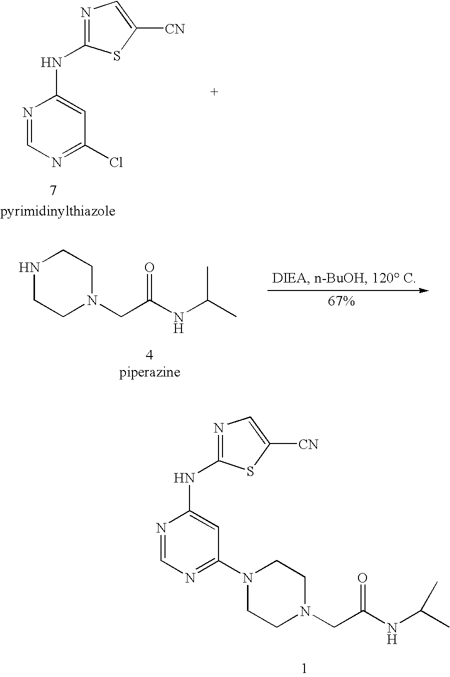

N-Isopropyl-2-piperazin-1-ylacetamide (4) (9.35 g, 50.5 mmol) is suspended in n-butanol (70 mL). 2-[(6-chloropyrimidin-4-yl)amino]-1,3-thiazole-5-carbonitrile (12.0 g, 50.5 mmol) and triethylamine (15.3 g, 151.5 mmol) are added and the mixture is then heated at 120° C. for 3 h. The reaction is cooled to room temperature. The solid is filtered, washed with n-butanol and ethyl ether, and then dried to afford 16.0 g (82%) of the final product 1. Purification is accomplished by first dissolving the 16.0 g in methylene chloride (250 mL) and methanol (60 mL). This solution is treated with decolorizing charcoal (24 g). The mixture is filtered through a filter-aid and the pad is washed with warm methylene chloride/methanol. The combined filtrates are evaporated to afford 13.77 g of the recovered product as a white solid. This is then purified on a silica column eluted with dichloromethane/MeOH/NH₄OH (95:5:0.5) to yield 10.45 g (65%) of 1:

¹H NMR (DMSO-d₆): 12.08(s, 1H); 8.43(s, 1H); 8.25(s, 1H); 7.54(d, 1H); 6.21(s, 1H); 3.90(m, 1H); 3.58(br s, 4H); 2.94(s, 2H); 2.50(br s, 4H); 1.08(d, 6H).

HPLC assay:

| Column: | Waters μ-Bondapack C18 | | | |
|---|---|---|---|---|
| Gradient: | Time | 0.1% H₃PO₄ | CH₃CN | |
| | | 0 min | 95% | 5% |
| | | 15 min | 5% | 95% |

Flow rate: 2.0 mL/min
Detection: 210 nm (254 nm can also be used)

Retention time

| | | |
|---|---|---|
| Diaminopyrimidine 1 | 4.546 min | 99.8 A % |

### Assays

Compounds prepared by the instant invention, as described in the Examples, were tested by the assays described below and were found to have kinase inhibitory activity. Other assays are known in the literature and could be readily performed by those of skill in the art (see, for example, Dhanabal et al., Cancer Res. 59:189-197; xin et al., J. Biol. Chem. 274:9116-9121; Sheu et al., Anticancer Res. 18:4435-4441; Ausprunk et al., Dev. Biol. 38:237-248; Gimbrone et al., J. Natl. Cancer Inst. 52:413-427; Nicosia et al., In Vitro 18:538-549).

I. VEGF Receptor Kinase Assay

VEGF receptor kinase activity is measured by incorporation of radio-labeled phosphate into polyglutamic acid, tyrosine, 4:1 (pEY) substrate. The phosphorylated pEY product is trapped onto a filter membrane and the incorporation of radio-labeled phosphate quantified by scintillation counting.

Materials

VEGF Receptor Kinase

The intracellular tyrosine kinase domains of human KDR (Terman, B. I. et al. Oncogene (1991) vol. 6, pp. 1677-1683.) and Flt-1 (Shibuya, M. et al. Oncogene (1990) vol. 5, pp. 519-524) were cloned as glutathione S-transferase (GST) gene fusion-proteins. This was accomplished by cloning the cytoplasmic domain of the KDR kinase as an in frame fusion at the carboxy terminus of the GST gene. Soluble recombinant GST-kinase domain fusion proteins were expressed in Spodoptera frugiperda (Sf21) insect cells (Invitrogen) using a baculovirus expression vector (pAcG2T, Pharmingen).

The other materials used and their compositions were as follows:

Lysis buffer: 50 mM Tris pH 7.4, 0.5 M NaCl, 5 mM DTT, 1 mM EDTA, 0.5% triton X-100, 10% glycerol, 10 mg/mL of each leupeptin, pepstatin and aprotinin and 1 mM phenylmethylsulfonyl fluoride (all Sigma).

Wash buffer: 50 mM Tris pH 7.4, 0.5 M NaCl, 5 mM DTT, 1 mM EDTA, 0.05% triton X-100, 10% glycerol, 10 mg/mL of each leupeptin, pepstatin and aprotinin and 1 mM phenylmethylsulfonyl fluoride.

Dialysis buffer: 50 mM Tris pH 7.4, 0.5 M NaCl, 5 mM DTT, 1 mM EDTA, 0.05% triton X-100, 50% glycerol, 10 mg/mL of each leupeptin, pepstatin and aprotinin and 1 mM phenylmethylsuflonyl fluoride.

10×reaction buffer: 200 mM Tris, pH 7.4, 1.0 M NaCl, 50 mM MnCl2, 10 mM DTT and 5 mg/mL bovine serum albumin (Sigma).

Enzyme dilution buffer: 50 mM Tris, pH 7.4, 0.1 M NaCl, 1 mM DTT, 10% glycerol, 100 mg/mL BSA.

10×Substrate: 750 μg/mL poly (glutamic acid, tyrosine; 4:1) (Sigma).

Stop solution: 30% trichloroacetic acid, 0.2 M sodium pyrophosphate (both Fisher).

Wash solution: 15% trichloroacetic acid, 0.2 M sodium pyrophosphate.

Filter plates: Millipore #MAFC NOB, GF/C glass fiber 96 well plate.

Method

A. Protein Purification

1. Sf21 cells were infected with recombinant virus at a multiplicity of infection of 5 virus particles/cell and grown at 27° C. for 48 hours.

2. All steps were performed at 4° C. Infected cells were harvested by centrifugation at 1000×g and lysed at 4° C. for 30 minutes with 1/10 volume of lysis buffer followed by centrifugation at 100,000×g for 1 hour. The supernatant was then passed over a glutathione Sepharose column (Pharmacia) equilibrated in lysis buffer and washed with 5 volumes of the same buffer followed by 5 volumes of wash buffer. Recombinant GST-KDR protein was eluted with wash buffer/10 mM reduced glutathione (Sigma) and dialyzed against dialysis buffer.

B. VEGF Receptor Kinase Assay

1) Add 5 μl of inhibitor or control to the assay in 50% DMSO.

2) Add 35 μl of reaction mix containing 5 μl of 10×reaction buffer, 5 141 25 mM ATP/10 μCi [³³P]ATP (Amersham), and 5 μl 10×substrate.

3) Start the reaction by the addition of 10 μl of KDR (25 nM) in enzyme dilution buffer.

4) Mix and incubate at room temperature for 15 minutes.

5) Stop by the addition of 50 μl stop solution.

6) Incubate for 15 minutes at 4° C.

7) Transfer a 90 μl aliquot to filter plate.

8) Aspirate and wash 3 times with wash solution.

9) Add 30 μl of scintillation cocktail, seal plate and count in a Wallac Microbeta scintillation counter.

II. HUMAN UMBILICAL VEIN ENDOTHELIAL CELL MIFOGENESIS ASSAY

Human umbilical vein endothelial cells (HUVECs) in culture proliferate in response to VEGF treatment and can be used as an assay system to quantify the effects of KDR kinase inhibitors on VEGF stimulation. In the assay described, quiescent HUVEC monolayers are treated with vehicle or test compound 2 hours prior to addition of VEGF or basic fibroblast growth factor (bFGF). The mitogenic response to VEGF or bFGF is determined by measuring the incorporation of [³H] thymidine into cellular DNA.

Materials

HUVECs: HUVECs frozen as primary culture isolates are obtained from Clonetics Corp. Cells are maintained in Endothelial Growth Medium (EGM; Clonetics) and are used for mitogenic assays described in passages 3-7 below.

Culture Plates: NUNCLON 96-well polystyrene tissue culture plates (NUNC #167008).

Assay Medium: Dulbecco's modification of Eagle's medium containing 1 g/mL glucose (low-glucose DMEM; Mediatech) plus 10% (v/v) fetal bovine serum (Clonetics).

Test Compounds: Working stocks of test compounds are diluted serially in 100% dimethylsulfoxide (DMSO) to 400-fold greater than their desired final concentrations.

Final dilutions to 1× concentration are made directly into Assay Medium immediately prior to addition to cells.

10×Growth Factors: Solutions of human VEGF165 (500 ng/mL; R&D Systems) and bFGF (10 ng/mL; R&D Systems) are prepared in Assay Medium.

10×[³H]Thyrnidine: [Methyl-3H]thymidine (20 Ci/mmol; Dupont-NEN) is diluted to 80 μCi/mL in low-glucose DMEM.

Cell Wash Medium: Hank's balanced salt solution (Mediatech) containing 1 mg/mL bovine serum albumin (Boehringer-Mannheim).

Cell Lysis Solution: 1 N NaOH, 2% (w/v) Na₂CO₃.

Method

1. HUVEC monolayers maintained in EGM are harvested by trypsinization and plated at a density of 4000 cells per 100 μL Assay Medium per well in 96-well plates. Cells are growth-arrested for 24 hours at 37° C. in a humidified atmosphere containing 5% CO₂.

2. Growth-arrest medium is replaced by 100 μL Assay Medium containing either vehicle (0.25% [v/v] DMSO) or the desired final concentration of test compound. All determinations are performed in triplicate. Cells are then incubated at 37° C. with 5% CO₂ for 2 hours to allow test compounds to enter cells.

3. After the 2-hour pretreatment period, cells are stimulated by addition of 10 μμL/well of either Assay Medium, 10×VEGF solution or 10×bFGF solution. Cells are then incubated at 37° C. and 5% CO₂.

4. After 24 hours in the presence of growth factors, 10×[³H]thymidine (10 μl/well) is added.

5. Three days after addition of [³H]thymidine, medium is removed by aspiration, and cells are washed twice with Cell Wash Medium (400 μl/well followed by 200 μL/well). The washed, adherent cells are then solubilized by addition of Cell Lysis Solution (100 μl/well) and warming to 37° C. for 30 minutes. Cell lysates are transferred to 7-mL glass scintillation vials containing 150 PL of water. Scintillation cocktail (5 ml/vial) is added, and cell-associated radioactivity is determined by liquid scintillation spectroscopy.

Based upon the foregoing assays the salts of Formula I are inhibitors of VEGF and thus are useful for the inhibition of angiogenesis, such as in the treatment of ocular disease, e.g., diabetic retinopathy and in the treatment of cancers, e.g., solid tumors. The instant salts inhibit VEGF-stimulated mitogenesis of human vascular endothelial cells in culture with IC50 values between 0.01-5.0 μM. These salts may also show selectivity over related tyrosine kinases (e.g., FGFR1 and the Src family; for relationship between Src kinases and VEGFR kinases, see Eliceiri et al., Molecular Cell, Vol. 4, pp.915-924, December 1999).

III. FLT-1 Kinase Assay

Flt-1 was expressed as a GST fusion to the Flt-I kinase domain and was expressed in baculovirus/insect cells. The following protocol was employed to assay compounds for Flt-1 kinase inhibitory activity:

1) Inhibitors were diluted to account for the final dilution in the assay, 1:20.

2) The appropriate amount of reaction mix was prepared at room temperature:

10× Buffer (20 mM Tris pH 7.4/0.1 M NaCl/mM DTT. final)

0.1M MnCl₂ (5 mM final)

pEY substrate (75 μg/mL)

ATP/[³³P]ATP (2.5 μM/L [Ci final)

BSA (500 μg/mL final).

3) 5 μL of the diluted inhibitor was added to the reaction mix. (Final volume of 5 μL in 50% DMSO). To the positive control wells, blank DMSO (50%) was added.

4) 35 μL of the reaction mix was added to each well of a 96 well plate.

5) Enzyme was diluted into enzyme dilution buffer (kept at 4° C.).

6) 10 μL of the diluted enzyme was added to each well and mix (5 nM final).

To the negative control wells, 10 μL 0.5 M EDTA was added per well instead (final 100 mM).

7) Incubation was then carried out at room temperature for 30 minutes.

8) Stopped by the addition of an equal volume (50 μL) of 30% TCA/0.1M Na pyrophosphate.

9) Incubation was then carried out for 15 minutes to allow precipitation.

10) Transfered to Millipore filter plate.

11) Washed 3× with 15% TCA/0.1M Na pyrophosphate (125 μL per wash).

12) Allowed to dry under vacuum for 2-3 minutes.

13) Dryed in hood for ˜20 minutes.

14) Assembled Wallac Millipore adapter and added 50 μL of scintillant to each well and counted.

IV. FLT-3 KINASE ASSAY

Flt-3 was expressed as a GST fusion to the Flt-3 kinase domain, and was expressed in baculovirus/insect cells. The following protocol was employed to assay compounds for Flt-3 kinase inhibitory activity:

1) Dilute inhibitors (account for the final dilution into the assay, 1:20) 2) Prepare the appropriate amount of reaction mix at room temperature.

10× Buffer (20 mM Tris pH 7.4/0.1 M NaCl/1 mM DTT final)

0.1M MnCl₂ (5 mM final)

pEY substrate (75 μg/mL)

ATP/[³³P]ATP (0.5 μM/L μCi final)

BSA (500 μg/mL final)

3) Add 5 μL of the diluted inhibitor to the reaction mix. (Final volume of 5 μL in 50% DMSO). Positive control wells—add blank DMSO (50%).

4) Add 35 μL of the reaction mix to each well of a 96 well plate.

5) Dilute enzyme into enzyme dilution buffer (keep at 4° C.).

6) Add 10 μL of the diluted enzyme to each well and mix (5-10 nM final). Negative control wells—add 10 μL 0.5 M EDTA per well instead (final 100 mM)

7) Incubate at room temperature for 60 minutes.

8) Stop by the addition of an equal volume (50 μL) of 30% TCA/0.1M Na pyrophosphate.

9) Incubate for 15 minutes to allow precipitation.

10) Transfer to Millipore filter plate.

11) Wash 3× with 15% TCA/0.1M Na pyrophosphate (125 μL per wash).

12) Allow to dry under vacuum for 2-3 minutes.

13) Dry in hood for ˜20 minutes.

14) Assemble Wallac Millipore adapter and add 50 μL of scintillant to each well and count.

What is claimed is:

1. A process for preparing substituted thiazolyl-amino pyrimidinyl piperazines of Formula I: 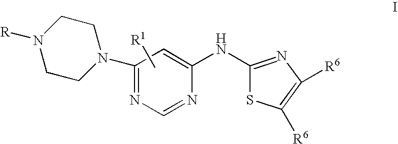
wherein
R is H, (C₁-C₆)alkyl, (C₀-C₆)alkyl-NR^{a}R^{b}, or (C₀-C₆)alkyl-C(O)N(Re)₂;
R¹ is H, or unsubstituted or substituted (C₁-C₆)alkyl;
R⁶ is independently selected from H, phenyl, halogen, CN, and pyridyl, said phenyl and pyridyl optionally substituted with one to three substituents selcted from R⁷;
R⁷ is independently selected from:
1) Oᵣ(C═O)SNR^{a}R^{b},
2) (C═O)ᵣOₛaryl,
3) (C═O)ᵣOₛ-heterocyclyl,
4) halogen,
5) OH,
6) O(C₁-C₃)perfluoroalkyl,
7) (C₁-C₃)perfluoroalkyl,
8) (C═O)ᵣOₛ(C1 C6)alkyl,
9) CO₂H,
10) CN,
11) (C₁-C₆)alkyl-NR^{a}R^{b}, and
12) (C₁-C₆)alkyl-heterocyclyl, wherein r and s are independently 0 or 1, and said aryl, heterocyclyl and alkyl are optionally substituted with one to three substituents selected from R^{d};
R^{a} and R^{b} are independently:
1) H,
2) (C═O)ᵣ(C₁-C₁₀)alkyl,
3) S(O)₂R^{c},
4) (C═O)ᵣheterocyclyl,
5) (C═O)^{r}aryl, and
6) CO₂RC,
wherein r is 0 or 1 and said alkyl, heterocyclyl, and aryl optionally substituted with one or more substituents selected from Rd;
R^{c} is independently selected from (C₁-C₆)alkyl, aryl, and heterocyclyl;
R^{d} is independently selected from:
1) (C═O)ᵣOS(C₁-C₁₀)alkyl, wherein r and s are independently 0 or 1, optionally substituted with up to three substituents selected from OH, (C₁-C₆)alkoxy, halogen, heterocyclyl, CN, oxo, N(Re)₂ and S(O)₂R^{c},
2) Oᵣ(C₁-C₃)perfluoroalkyl,
3) (C₀-C₆)alkylene-S(O)ₘR^{c}, wherein m is 0, 1, or 2,
4) OH,
5) halo,
6) CN,
7) (C₀-C₆)alkylene-aryl, optionally substituted with up to three substituents selected from R^{e},
8) (C₀-C₆)alkylene-heterocyclyl, optionally substituted with up to three substituents selected from R^{e},
9) C(O)R^{c},
10) CO₂R^{c},
11) C(O)H,
12) N(R^{e})₂, and
13) CO₂H;
R^{e} is independently selected from:
1) H,
2) (C₁-C₆)alkyl, optionally substituted with one or more substituents selected from OH, heterocyclyl, (C₁-C₆)alkoxy, halogen, CN, oxo, N(R^{f})₂ and S(O)₂R^{c},
3) aryl, optionally substituted with one or more substituents selected from OH, heterocyclyl, (C₁-C₆)alkoxy, halogen, CN, N(R^{f})₂ and S(O)₂R^{c},
4) heterocyclyl, optionally substituted with one or more substituents selected from OH, heterocyclyl, (C₁-C₆)alkoxy, halogen, CN, oxo, N(R^{f})₂ and S(O)₂R^{c}, and
5) S(O)₂R^{c};
said heterocycle optionally substituted with one or more substituents selected from OH, (C₁-C₆)alkoxy, halogen, CN, oxo, N(R^{f})₂ and S(O)₂R^{c}; and
R^{f} is independently selected from H, aryl and (C₁-C₆)alkyl;
which comprises the steps of:
a) reacting a compound of Formula II 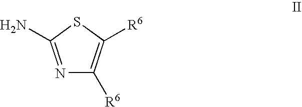
with a compound of Formula III 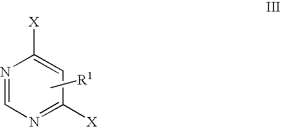
(wherein X is halo), to provide a compound of Formula IV 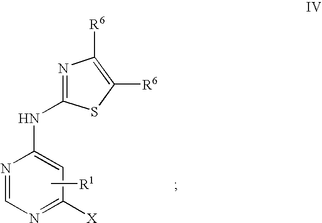
b) reacting the compound of Formula IV with a compound of Formula V 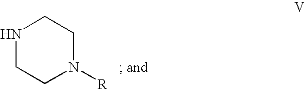
c) isolating the compound of Formula I.

2. The process according to claim 1 which comprises the steps of:
a) adding the compounds of Formula II and Formula III and a phosphate to a first solvent;
b) isolating the compound of Formula IV;
c) adding the compound of Formula IV and a trialkylamine to a mixture of the compound of Formula V in a second solvent; and
d) isolating the compound of Formula I.

3. The process according to claim 2 wherein the first solvent is selected from unchlorinated or chlorinated hydrocarbons, nitriles, ethers, polar aprotic solvents or mixtures thereof.

4. The process according to claim 2 wherein the second solvent is selected from water, alcohols, unchlorinated or chlorinated hydrocarbons, nitrites, ketones, ethers, polar aprotic solvents or mixtures thereof.

5. The process according to claim 2 wherein the unsubstituted or substituted amine is selected from unsubstituted or substituted arylamine, unsubstituted or substituted heteroarylamine, unsubstituted or substituted C₁-C₆ alkylamines, ammonia, H₂N-R^{a}C(O)OR and H₂N-R^{a}SR.

6. The process according to claim 1 which comprises the steps of:
a) adding the compounds of Formula II and Formula III and a carbonate to a first solvent;
b) isolating the compound of Formula IV;
c) adding the compound of Formula IV and a trialkylamine to a mixture of the compound of Formula V in a second solvent; and
d) isolating the compound of Formula I.

7. A process for preparing 2-(4-{6-[(5-cyano-1,3-thiazol-2-yl)amino]pyrimidin-4-yl}piperazin-1-yl)-N-isopropylacetamide 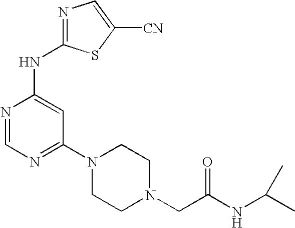
which comprises the steps of:
a) adding 2-amino-5-cyanothiazole, dichloropyrmidine, and K₃PO₄ to DMF to provide 2-[(6-chloropyrimidin-4-yl)amino]-1,3-thiazole-5-carbonitrile;
b) adding 2-[(6-chloropyrimidin-4-yl)amino]-1,3-thiazole-5-carbonitrile and triethylamine to N-Isopropyl-2-piperazin-1-ylacetamide in n-butanol; and
c) isolating 2-(4-{6-[(5-cyano-1,3-thiazol-2-yl)amino]pyrimidin-4-yl} piperazin-1-yl)-N-isopropylacetamide.
